(19) United States
(12) Patent Application Publication
(10) Pub. No.: US 2025/0136487 A1
(43) Pub. Date: May 1, 2025

(54) NUTRIENT SUPPLEMENTED CARBONIZED AEROGELS

(71) Applicant: B. G. NEGEV TECHNOLOGIES AND APPLICATIONS LTD., Beer Sheva (IL)

(72) Inventors: Ariel KUSHMARO, Beer Sheva (IL); Danit Lisa KARSAGI, Beer Sheva (IL)

(73) Assignee: B. G. NEGEV TECHNOLOGIES AND APPLICATIONS LTD., Beer Sheva (IL)

(21) Appl. No.: 18/834,692

(22) PCT Filed: Feb. 7, 2023
(86) PCT No.: PCT/IL2023/050130

§ 371 Date: Jul. 31, 2024

Related U.S. Application Data
(60) Provisional application No. 63/307,173, filed on Feb. 7, 2022.

(51) Int. Cl. C02F 3/34; B01J 20/20; B01J 20/28; B01J 20/30; B09C 1/00; B09C 1/08; B09C 1/10; C01B 32/05; C02F 1/28; C02F 3/00; C02F 3/10; C02F 101/32; C02F 103/00; C02F 103/06; C02F 103/08; C12N 1/20

(52) U.S. Cl. CPC C02F 3/344; B01J 20/20; B01J 20/28023; B01J 20/28047; B01J 20/28061; B01J 20/3078; B09C 1/08; B09C 1/10; C01B 32/05; C02F 1/283; C02F 3/106; C02F 3/348; C12N 1/20; B09C 1/002; B09C 2101/00; C02F 2003/001; C02F 2101/32; C02F 2103/007; C02F 2103/06; C02F 2103/08; C02F 2305/06

(57) ABSTRACT

A carbon-based aerogel doped or enriched with nitrogen and phosphorus is provided. Further, kits comprising the aerogel and oil-degrading bacteria are provided. Use of the kits for at least partially degrading a crude oil or a crude oil fraction is also provided.

### CROSS REFERENCE TO RELATED APPLICATIONS

This application claims the benefit of priority to U.S. Provisional Patent Application No. 63/307,173 filed Feb. 7, 2022 entitled “Nutrient Supplemented Carbonized Aerogels”, the contents of which are incorporated herein by reference in their entirety.

### FIELD OF THE INVENTION

The present invention is in the field of material science and electrocatalysis.

### BACKGROUND OF THE INVENTION

Oil spills at sea are some of the most disastrous of anthropogenic pollution events. With surging oil demand and sophisticated technological developments, exploration and production has accelerated in recent years. Crude petroleum and other hydrocarbons components are highly toxic to microorganisms, plants and animals including humans. The biological effects of gas and oil use, in turn affect ecosystem structures and balance, amplifying the importance of developing an adequate and immediate response.

Although it possible to adsorb crude oil spills from the water body, using various adsorbents, the adsorbed oil will be inevitably retained in the water column and in the sediment resulting in bio-accumulation and persistence in the habitat. Natural biodegradation of this oil is slow and occurs by natural microbial communities that allows for the conversion of the hazardous constituents into forms that are less toxic. Therefore, there is a long felt need to develop efficient means for inducing crude oil degradation, to substantially eliminate oil spills from the environment.

### SUMMARY OF THE INVENTION

According to one aspect, there is provided a composition comprising a carbon aerogel and a material bound to the carbon aerogel; wherein the material comprises a nitrogen-containing material and a phosphorus-containing material; wherein the combined content of nitrogen and phosphorus within the composition is at least 1 atomic percentage (at %).

In some embodiments, the aerogel is characterized by carbon content between 70 and 95 at %.

In some embodiments, the material further comprises an additional element selected from Fe, Zn, S, Na, Ca, Ti, and Mg, including any salt, any oxide, any complex, or any combination thereof.

In some embodiments, the additional element within the composition is present at a concentration of 0.1 to 10 at %.

In some embodiments, the carbon aerogel is in a form of a matrix comprising a plurality of carbon fibers.

In some embodiments, the material is covalently or physically bound to the plurality carbon fibers.

In some embodiments, the carbon aerogel is characterized by: (i) a nitrogen content of between 1 and 10 at %; (ii) a phosphorus content of between 1 and 10 at %, or both (i) and (ii).

In some embodiments, the carbon aerogel is further characterized by an oxygen content of between 5 and 20 at %.

In some embodiments, the carbon aerogel is characterized by a water contact angle of between 12° and 140°.

In some embodiments, the carbon aerogel is characterized by porosity of between 95 and 99%.

In some embodiments, the composition further comprising bacterial cells attached to the carbon aerogel.

In some embodiments, the bacterial cells are oil degrading bacteria.

In some embodiments, the oil degrading bacteria comprise one or more bacterial species selected from genera Pseudomonas and Brucella.

In some embodiments, the bacterial cells comprise a biofilm.

In some embodiments, the carbon aerogel is capable of absorbing at least 100% of a water immiscible organic compound by weight of the carbon aerogel.

In some embodiments, the water immiscible organic compound is selected from crude oil, a crude oil fraction and a water immiscible organic solvent.

In some embodiments, the carbon aerogel is characterized by BET surface area of between 150 and 300 m2/g.

In another aspect, there is provided a method for removing a crude oil or a crude oil fraction, comprising applying the composition of the invention to the crude oil or the crude oil fraction, thereby removing the crude oil or the crude oil fraction.

In some embodiments, the composition comprises oil degrading bacteria attached to the carbon aerogel.

In some embodiments, the applying is at an amount sufficient for substantially absorbing the crude oil or the crude oil fraction.

In some embodiments, the crude oil or the crude oil fraction is present in soil or water.

In another aspect, there is provided a method for degrading a crude oil or a crude oil fraction, comprising contacting the crude oil or the crude oil fraction with a composition comprising a carbon aerogel and a material bound to the carbon aerogel; wherein the material comprises a nitrogen-containing material and a phosphorus-containing material; wherein a combined content of nitrogen and phosphorus within the carbon aerogel is at least 1 at %; and wherein the composition comprises oil degrading bacteria attached to the carbon aerogel.

In some embodiments, the combined content of nitrogen and phosphorus is sufficient for inducing increased degradation of the crude oil or of the crude oil fraction, as compared to a similar composition devoid of the material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B are graphs presenting TPD analysis of nutrient containing pulp. 1A. Full spectra and 1B. Co2 and H2O subtracted.

FIGS. 2A-2C are scanning electron microscopy (SEM) micrographs presenting 550× magnified images of 2A. cellulose paper, 2B. carbonized aerogel, 2C. nutrient supplemented carbonized aerogel.

FIGS. 3A-3B are images presenting water drops on hydrophobic aerogel products, 3a. Carbonized aerogel (CA) and 3B. Nutrient supplemented carbonized aerogel (NDCA).

FIG. 4 is a bar graph shoeing adsorption capacities of CA and NDCA.

FIGS. 5A-5B are SEM micrographs presenting bacterial adhesion to 5A. CA fibers and 5B. NDCA fibers.

FIGS. 6A-6C. Enhanced hydrocarbon biodegradation using nutrient supplemented carbonized aerogel inoculated with 6A. P. Aeruginosa & O. intermedium 6B. P. Aeruginosa 6C. O. intermedium.

FIGS. 7A-7C. Hydrocarbon biodegradation using P. Aeruginosa & O. intermedium with 7A. Nutrient supplemented carbonized aerogel. 7B. Carbonized aerogel 7C. No aerogel.

### DETAILED DESCRIPTION OF THE INVENTION

According to some embodiments, the present invention provides a porous carbon material enriched or supplemented with bacterial nutrients comprising nitrogen and phosphorus, wherein the porous carbon material is in a form of a carbon aerogel. According to some embodiments, the present invention provides the porous carbon material as a scaffold for attachment of bacterial cells. In some embodiments, the porous carbon material of the invention enables enhanced bacterial proliferation, and/or activity, compared to a similar non-enriched porous carbon material. According to some embodiments, the present invention provides a method for at least partially degrading a crude oil or a fraction thereof, by contacting the crude oil or fraction thereof with the porous carbon material of the invention comprising oil-degrading bacteria attached thereto.

### The Composition

According to one aspect of the invention, there is provided a carbon aerogel doped or enriched with a nitrogen-containing material and with a phosphorus-containing material, wherein the combined content of nitrogen and phosphorus within the aerogel is at least 1 atomic percentage (at %).

According to another aspect of the invention, there is provided a carbon aerogel in contact with or bound to a nitrogen-containing material and to a phosphorus-containing material, wherein the combined content of nitrogen and phosphorus within the aerogel is at least 1 at %.

In some embodiments, there is provided a composition comprising the carbon aerogel in contact with or bound to a material comprising the nitrogen-containing material and a phosphorus-containing material; and wherein a combined content of nitrogen and phosphorus within the composition is at least 1 at %.

As used herein, the term “carbon aerogel” encompasses an aerogel consisting essentially of carbon material and further comprises the nitrogen-containing material and the phosphorus-containing material bound to or in contact with the carbon material. The term “aerogel” is well-known in the art and refers to a highly porous solid material. Thus, the terms “carbon aerogel” and “aerogel” are used herein interchangeably.

In some embodiments, the carbon aerogel of the invention consists essentially of the carbon aerogel and the nitrogen-containing material and the phosphorus-containing material. In some embodiments, the carbon aerogel consists essentially of is composed the carbon aerogel, and the bacterial nutrient comprising the nitrogen containing material, the phosphorus-containing material, and optionally the additional element (e.g., Fe, Zn, S, Na, Ca, Ti, and Mg, including any salt, any oxide, any complex, or any combination thereof). In some embodiments, at least 70%, at least 80%, at least 90%, at least 95%, at least 97%, at least 99%, or between 70 and 100%, between 80 and 100%, between 90 and 100%, between 90 and 99%, between 92 and 99%, between 95 and 99% by weight of the carbon aerogel, or of the composition of the invention consists of the aerogel, and the bacterial nutrient comprising the nitrogen containing material, the phosphorus-containing material, and optionally the additional element, including any range between.

In some embodiments, at least 70%, at least 80%, at least 90%, at least 95%, at least 97%, at least 99%, or between 70 and 100%, between 80 and 100%, between 90 and 100%, between 90 and 99%, between 92 and 99%, between 95 and 99% by weight of the composition of the invention consists of the aerogel, the bacterial nutrient comprising the nitrogen containing material, the phosphorus-containing material, and optionally the additional element, and of oil-degrading bacteria, including any range between.

In some embodiments, the aerogel is a continuous substantially homogenous solid material. In some embodiments, the aerogel is characterized by at least one dimension (e.g. length dimension, width dimension, height dimension, and/or cross-section, including any combination thereof) of at least 1 um, at least 10 um, at least 100 um, at least 1 mm, at least 1 cm, at least 10 cm, between 0.1 mm and 1 m, between 1 mm and 1 m, between 1 mm and 50 cm, between 1 and 50 cm, between 1 and 30 cm, including any range between.

In some embodiments, the chemical composition of the substantially homogenous solid material varies by not more than 20%, not more than 10%, not more than 5%, not more than 1%, within the entire volume of the solid material. The term chemical composition as used herein, refers to the elemental content of the solid material (e.g., atomic percentage of C, and of additional elements such as O, N, and P).

In some embodiments, the substantially homogenous solid material is characterized by the same physical structure (i.e., a porous fibrous matrix) and/or physical properties (e.g., BET, water contact angle, etc.) within the entire volume of the solid material. In some embodiments, the porosity of the substantially homogenous solid material varies by not more than 20%, not more than 10%, not more than 5%, not more than 1%, within the entire volume of the solid material.

In some embodiments, the aerogel (and/or the composition) of the invention is devoid of an additional material (such as CNT, graphene, fullerene, carbon black, metal, composite material). In some embodiments, the aerogel (and/or the composition) of the invention is devoid of a polymer which is not a carbon fiber. In some embodiments, the aerogel of the invention is substantially devoid of an inorganic salt, and/or of an inorganic polymer. In some embodiments, the aerogel (and/or the composition) of the invention is devoid of a material which is not a carbon fiber. In some embodiments, at least 80%, at least 90%, at least 93%, at least 95%, at least 97%, at least 99%, or between 80 and 99%, between 80 and 100%, between 85 and 99%, between 90 and 100%, between 95 and 99% by weight of the aerogel is composed of carbon fibers, including any range between.

In some embodiments, the composition of the invention consists essentially of the carbon aerogel bound to the material, as disclosed herein.

In some embodiments, the aerogel is a solid. In some embodiments, the aerogel is in a form of or comprises a fibrous matrix. In some embodiments, the fibrous matrix is a porous matrix. In some embodiments, the fibrous matrix (or aerogel) is characterized by a porosity between 95 and 99%, between 90 and 99%, between 95 and 96%, between 95 and 97%, between 95 and 98%, between 96 and 99%, between 96 and 98%, between 97 and 99%, between 96 and 98%, including any range between. The term “porosity” as used herein, refers to the relative volume of the void space relative to the entire volume of the aerogel.

In some embodiments, the fibrous matrix (or aerogel) is characterized by BET surface area of at most 300 m²/g, at most 280 m²/g, at most 260 m²/g, at most 240 m²/g, at most 220 m²/g, between 150 and 300 m²/g, between 200 and 300 m²/g, between 150 and 250 m²/g, between 200 and 250 m²/g, between 200 and 230 m²/g, including any range between.

In some embodiments, the aerogel or the composition of the invention is characterized by BET surface area of between 200 and 230 m²/g, including any range between.

In some embodiments, the fibrous matrix comprises or consists essentially of carbon fibers. In some embodiments, the carbon fibers consist essentially of carbon atoms (e.g., arranged in a form of fused polycyclic aromatic rings). In some embodiments, the fibrous matrix is a carbonized matrix. In some embodiments, the fibrous matrix and/or the aerogel of the invention consist essentially of carbon material, of nitrogen-containing material and of a phosphorus-containing material. As used herein, the term “carbon material” refers to carbon containing structures.

In some embodiments, the carbon material in the aerogel of the invention is an activated carbon. In some embodiments, the fibrous matrix and/or the aerogel is a pyrolyzed cellulose material. In some embodiments, the fibrous matrix and/or the aerogel is a carbonized material. In some embodiments, the fibrous matrix and/or the aerogel is prepared by pyrolysis of a composition comprising (i) a carbon precursor, (ii) a nitrogen-containing material precursor (e.g. ammonia, urea, guanidine including any combination and any salt thereof) and (iii) a phosphorus-containing material precursor (a phosphate salt including any di-, tri-, or polyphosphate, a phosphite salt, phosphoric acid, phosphorus acid, or any combination thereof). In some embodiments, the carbon precursor comprises a polysaccharide. In some embodiments, the carbon precursor comprises cellulose or cellulose-based material, such as paper, carton, recycled paper, or any fibrous solid material derived from a plant matter.

In some embodiments, the carbon content of the aerogel (or of the composition) of the invention is between 70 and 95 at %, between 70 and 94 at %, between 70 and 93 at %, between 75 and 95 at %, between 80 and 95 at %, between 70 and 90 at %, between about 80 and about 95 at %, between 80 and 93 at %, between 80 and 90 at %, between about 85 and about 90 at %, or between about 85 and about 88 at %, including any range between.

In some embodiments, the oxygen content of the aerogel (or of the composition) of the invention is between 5 and 20 at %, between 5 and 15 at %, between about 10 and about 15 at %, between about 8 and about 15 at %, including any range between. In some embodiments, molar ratio between C and O within the aerogel (or in the composition) of the invention is between 7:1 and 15:1, between 7:1 and 9:1, between 7:1 and 10:1, between 8:1 and 15:1, between 8:1 and 10:1, between 8:1 and 15:1, between 8:1 and 12:1, including any range between. In some embodiments, the O content and the C content of the aerogel is identical with the O content and the C content of the carbon fibers.

In some embodiments, the carbon fibers consist essentially of carbon atoms, oxygen atoms, and hydrogen atoms, and optionally comprises one or more impurities such as a salt (e.g., alkaline metal cations or a salt comprising thereof), metal oxide, metal cation, or any combination thereof. In some embodiments, the w/w concentration of one or more impurities within the carbon fibers is between 10 ppm and 10%, between 10 ppm and 8%, between 10 ppm and 1%, between 10 ppm and 0.1%, between 100 ppm and 0.1%, between 0.1% and 10%, including any range between.

In some embodiments, the oxygen atoms are in a form of an oxygen-based material comprising an oxide, an oxygen containing organic matter (e.g., a heterocyclic aromatic or aliphatic optionally fused ring, such as furan, dioxane, oxane, pyran, etc.), or a functional group containing an oxygen atom bound to the aromatic ring (e.g., a carbonyl, an oxy group, a hydroxy group, an ether, etc.). In some embodiments, the oxygen atoms are covalently bound to the carbon atoms (e.g., hydroxy, oxy, carboxy, or carbonyl group; or in a form of a cyclic or linear heteroalkyl). In some embodiments, the oxygen atoms are in a form of oxides or salts and are physically adsorbed to the carbon fibers.

As used herein, the term “matrix” refers to one or more porous layers of carbon fibers that are randomly, and/or under certain order or control, distributed therewithin. Matrix may further include any materials incorporated within and/or interposed between the layers. In some embodiments, the matrix comprises randomly oriented carbon fibers. In some embodiments, each carbon fiber within the matrix is in contact with at least one additional carbon fiber. In some embodiments, the carbon fibers are randomly distributed within the matrix, to obtain a three-dimensional mesh structure comprising a void space between the fibers. In some embodiments, the carbon fibers are randomly distributed within the matrix thus forming a plurality of pores (or void space). In some embodiments, the matrix is an intertwined matrix composed of randomly distributed carbon fibers. In some embodiments, the matrix is substantially devoid of aligned or oriented carbon fibers. In some embodiments, the matrix is substantially devoid of carbon fibers aligned or oriented in a specific direction.

In some embodiments, the entire aerogel is in a form of a continuous matrix comprising a plurality of pores. In some embodiments, the plurality of pores is characterized by an average pore size between 1 and 500 um, between 10 and 100 um, between 10 and 500 nm, between 10 and 200 um, between 10 and 300 um, including any range in between.

In some embodiments, the carbon fibers are characterized by an average cross-section between 1 and 100 um, between 1 and 50 um, between 10 and 100 um, between 10 and 50 um, including any range between.

In some embodiments, the nitrogen-containing material and a phosphorus-containing material each independently are bound to the carbon fibers of the matrix. In some embodiments, the nitrogen-containing material and a phosphorus-containing material each independently are bound to the outer surface of the carbon fibers (i.e., a surface facing the void space). In some embodiments, the nitrogen-containing material and a phosphorus-containing material each independently are bound to or incorporated within the aerogel of the invention. In some embodiments, the nitrogen-containing material and a phosphorus-containing material each independently are adsorbed (e.g., physisorbed) on or within the aerogel. In some embodiments, the nitrogen-containing material and a phosphorus-containing material each independently are adsorbed (e.g., physisorbed) on or within the carbon fibers. In some embodiments, the carbon fibers or the aerogel of the invention is doped or enriched with the nitrogen-containing material and with the phosphorus-containing material.

In some embodiments, the term “bound” refers to any non-covalent bond or interaction, such as electrostatic bond, dipole-dipole interaction, Van-der-walls' interaction, ionotropic interaction, hydrogen bond, hydrophobic interactions, pi-pi stacking, London forces, etc. In some embodiments, the non-covalent bond or interaction is a stable bond or interaction, wherein stable encompasses that at least 80%, at least 90%, at least 95% of the initial content of the nitrogen-containing material and of the phosphorus-containing material is retained upon contacting the aerogel with water at a temperature up to 30° C. In some embodiments, the nitrogen-containing material and of the phosphorus-containing material is substantially released upon contacting the aerogel of the invention with bacteria. Thus, a skilled artisan will appreciate that the nitrogen-containing material and of the phosphorus-containing material is bioavailable.

In some embodiments, the term “enriched” as used herein refers to an aerogel having a greater N and P content compared to similar non-enriched aerogel (i.e. aerogel obtained by pyrolysis of cellulose based mater without addition of urea and phosphoric acid). In some embodiments, the combined N and P content of the enriched aerogel of the invention is greater than the combined N and P content of the non-enriched aerogel by at least 2 times, at least 5 times, at least 10 times, at least 100 times, at least 1000 times, at least 100.000 times, or between 2 times and 100.000 times, including any range between.

In some embodiments, the carbon material is nitrogen and phosphorus doped material. In some embodiments, the nitrogen-containing material and the phosphorus-containing material are embedded into the carbon fibers of the aerogel.

In some embodiments, the nitrogen-containing material, and a phosphorus-containing material are each independently covalently bound to the carbon fibers of the matrix. In some embodiments, the phosphorus-containing material is covalently bound to the residues of hydroxy motifs in the carbon fibers. In some embodiments, the phosphorus-containing material is bound to carbocyclic (aromatic or aliphatic) ring(s). In some embodiments, the phosphorus-containing material is or comprises phosphate (inorganic and/or organic phosphate), phosphite, phosphoester, phosphodiester, phosphine, or any combination thereof. In some embodiments, the phosphorus-containing material comprises organic and/or inorganic compounds (e.g., phosphate salt). In some embodiments, the phosphate of the phosphorus-containing material is covalently bound to the carbon material (or carbon fibers) via hydroxy or oxy groups.

In some embodiments, the nitrogen-containing material is in a form of a heterocyclic ring (aliphatic or aromatic). In some embodiments, the nitrogen-containing material is in a form of inorganic material, such as ammonium salt, urea, nitrate salt, or any combination thereof. In some embodiments, the nitrogen-containing material is in a form of functional groups bound to the carbocyclic rings of the carbon fibers. In some embodiments, functional groups are selected from an amine group, an imine group, nitro, cyano, amide, guanidine, urea, or any combination thereof. The exact content of the functional groups within the nitrogen-containing material and within the phosphorus-containing material (e.g., phosphoesters, phosphine, amine group, an imine group, etc.) present within the aerogel of the invention can be determined via spectroscopic methods such as IR, RAMAN, XPS, or any combination thereof. In some embodiments, the nitrogen-containing material and phosphorus-containing material are covalently bound or complexed to each other, such as in a form of monoammonium monohydrogen phosphate.

In some embodiments, the w/w concentration of the nitrogen-containing material, or of the phosphorus-containing material within the aerogel is each independently between 0.5 and 10%, between 1 and 10%, between 0.5 and 5%, between 1 and 5%, between 1 and 4%, between 2 and 5%, between 2 and 10%, including any range between.

In some embodiments, a combined w/w concentration of the nitrogen-containing material and of the phosphorus-containing material within the aerogel is each independently between 1 and 20%, between 5 and 15%, between 5 and 20%, between 5 and 10%, between 3 and 7%, between 3 and 10%, including any range between.

In some embodiments, the N content of the aerogel of the invention is between 0.5 and 10 at %, between 1 and 10 at %, between 1 and 5 at %, between 2 and 10 at %, between 2 and 7 at %, between 2 and 6 at %, including any range between.

In some embodiments, the P content of the aerogel of the invention is between 0.5 and 10 at %, between 1 and 10 at %, between 1 and 5 at %, between 2 and 10 at %, between 2 and 7 at %, between 2 and 6 at %, including any range between.

In some embodiments, the combined N and P content of the aerogel of the invention is at least 1 at %, at least 2 at %, at least 3 at %, at least 5 at %, at least 6 at %, between 1 and 20 at %, between 2 and 10 at %, between 5 and 15 at %, between 5 and 20 at %, between 5 and 10 at %, including any range between.

In some embodiments, molar ratio between N and P within the aerogel of the invention is between 3:1 and 1:3, between 3:1 and 1:3, between 2:1 and 1:1, between 2:1 and 1:2, between 1.5:1 and 1:1, between 3:1 and 1.5:1, including any range between. In some embodiments, the elemental content of the aerogel is determined by EDS.

In some embodiments, the aerogel of the invention is substantially devoid of metal (e.g., a metal content below 0.1%, below 0.01%, below 0.001% or any range between). In some embodiments, the aerogel of the invention further comprises an additional element selected from Fe, Zn, S, Na, Ca, and Mg, including any salt, any cation, any oxide, any complex, or any combination thereof. In some embodiments, the additional element comprises a metal cation, a salt thereof, an oxide thereof, or any combination thereof) wherein the metal is essential for bacterial proliferation (e.g., a transitional metal cation, such as Fe, Cu, Co, Ti.

In some embodiments, a content of the additional element within the aerogel is between 0.1 and 10 at %, between 0.1 and 5 at %, between 0.1 and 1 at %, between 0.01 and 1% w/w including any range between.

In some embodiments, the aerogel of the invention is characterized by a water contact angle of at least 90° and between about 90 and 150°, between 10° and 150°, between 10° and 140°, between 12° and 140°, between 12° and 135°, between 11° and 120°, between 115 and 125°, between 12° and 130°, between 125 and 135°, between 13° and 140°, between 135 and 145°, including any range in between.

In some embodiments, the outer surface of the aerogel is hydrophobic. In some embodiments, the aerogel has low capability to absorb water. In some embodiments, the aerogel is characterized a water absorption capacity of at most 150%, at most 130%, at most 110%, at most about 100%, by weight of the aerogel, including any range in between.

In some embodiments, the aerogel is characterized by an absorption capability towards a water immiscible organic compound of at least 100%, at least 150%, at least 200%, at least 250%, at least 500%, at least 750%, at least 1000% by weight of the aerogel, including any range in between. In some embodiments, the aerogel is characterized by an absorption capability of water immiscible organic compounds by weight of the aerogel of between 100 and 1500%, between 100 and 250%, between 250% and 500%, between 500 and 750%, between 750 and 1000%, between 1000 and 1250%, between 1250 and 1500%, including any range in between.

In some embodiments, the water immiscible organic compounds are selected from crude oil, crude oil fraction and water immiscible organic solvent (e.g., pentane, hexane, heptane, octane, chlorinated hydrocarbons, aromatic solvents, etc.).

In some embodiments, the aerogel of the invention is a cell scaffold. In some embodiments, the aerogel of the invention promotes cell attachment thereto. In some embodiments, the aerogel of the invention is configured to support cell growth, cell proliferation, cell metabolism, and/or formation of biofilm on or within the aerogel. In some embodiments, the cell is a bacterial cell.

In some embodiments, the aerogel of the invention further comprises one or more bacterial cells attached thereto. In some embodiments, attachment is by physical interaction, between the bacteria cell wall and the aerogel. In some embodiments, the one or more bacterial cells comprises a plurality of cells. In some embodiments, the plurality of cells forms a biofilm on at least one surface of the aerogel. In some embodiments, the one or more bacterial cells are isolated bacteria.

In some embodiments, the bacterial cell is attached to the aerogel of the invention. In some embodiments, the bacterial cell comprises a plurality of bacterial cells in a form of a biofilm. In some embodiments, the bacteria cell is an oil degrading bacterium. In some embodiments, the term “oil degrading bacterium” and the term “petroleum hydrocarbon-degrading bacterium” are used herein interchangeably.

In some embodiments, the oil-degrading bacterium comprises a single bacterial specie. In some embodiments, the oil-degrading bacterium comprises a plurality of bacterial species. In some embodiments, the oil-degrading bacterium comprises one or more bacterial species from phylum Pseudomonadota. In some embodiments, the oil-degrading bacterium comprises at least one specie selected from class Alphaproteobacteria, Betaproteobacteria and Gammaproteobacteria. In some embodiments, the oil-degrading bacterium comprises at least two species selected from class Alphaproteobacteria, Betaproteobacteria and Gammaproteobacteria. In some embodiments, the oil-degrading bacterium comprises one or more species selected from genera Achromobacter, Acinetobacter, Alkanindiges, Alteromonas, Arthrobacter, Burkholderia, Dietzia, Enterobacter, Kocuria, Marinobacter, Mycobacterium, Pandoraca, Pseudomonas, Staphylococcus, Streptobacillus, Streptococcus, and Rhodococcus. In some embodiments, the oil-degrading bacterium comprises at least two species selected from genera Achromobacter, Acinetobacter, Alkanindiges, Alteromonas, Arthrobacter, Burkholderia, Dietzia, Enterobacter, Kocuria, Marinobacter, Mycobacterium, Pandoraca, Pseudomonas, Staphylococcus, Streptobacillus, Streptococcus, and Rhodococcus. In some embodiments, the oil-degrading bacterium comprises two or more species of genera Pseudomonas and Brucella. In some embodiments, the oil-degrading bacterium comprises a single species from genus Pseudomonas or Brucella. In some embodiments, the oil-degrading bacterium comprises Pseudomonas Aeruginosa, Ochrobactrum, intermedium, or both.

In some embodiments, provided a composition comprising the bacterial cells attached to the aerogel of the invention, wherein the loading of the bacteria is between 100 and 100,000,000 CFU/cm², between 1000 and 100,000,000 CFU/cm², between 100,000 and 100,000,000 CFU/cm², between 1,000,000 and 100,000,000 CFU/cm², between 100,000 and 10,000,000 CFU/cm², between 100.000 and 100,000,000,000 CFU/cm², between 100.000 and 1,000,000,000 CFU/cm², between 100.000 and 10,000,000,000 CFU/cm², including any range between.

In some embodiments, the aerogel of the invention enhances at least one of: attachment, proliferation, metabolism, and oil degrading activity of the oil degrading bacterium attached thereto, wherein “enhances” is relative to a similar non-enriched carbon aerogel. In some embodiments, the oil degrading bacteria attached to the aerogel of the invention are characterized by enhanced oil degradation rate, compared to the same bacteria attached to a similar non-enriched carbon aerogel, wherein enhanced is by at least 10%, at least 50%, at least 30%, at least 70%, at least 200%, at least 300%, at least 500%, including any range between.

In another aspect, there is provided a kit comprising the aerogel of the invention and a plurality of oil degrading bacteria. In some embodiments, each of the aerogel the plurality of oil degrading bacteria is stored in separate containers or compartments. In some embodiments, each of the compartments is in a form of an airtight and/or moisture tight package. In some embodiments, the plurality of oil degrading bacteria within the kit comprises of a single bacterial specie or a plurality of species. In some embodiments, the plurality of oil degrading bacteria within the kit are isolated bacteria. In some embodiments, the plurality of oil degrading bacteria within the kit are lyophilized bacteria. In some embodiments, the plurality of oil degrading bacteria within the kit are isolated and lyophilized bacteria. In some embodiments, the ratio between the plurality of oil degrading bacteria and the aerogel is so as to obtain a bacterial load, as disclosed hereinabove.

In another aspect, there is provided a method for treating a location contaminated with a crude oil or a crude oil fraction, comprising applying the composition or the kit of the invention to the location, wherein the composition comprises the oil-degrading bacteria attached to the aerogel of the invention. In some embodiments, the method is for inducing at least partial degradation of the crude oil or the crude oil fraction.

In some embodiments, applying the composition is at an amount sufficient for substantially absorbing the crude oil or the crude oil fraction. In some embodiments, sufficient amount refers to a w/w ratio between the aerogel and the oil crude or oil crude fraction of between 1:1 and 1:10,000, between 1:1 and 1:1,000, between 1:1 and 1:100, between 1:1 and 1:10, between 1:1 and 1:5, between 1:1 and 1:25, between 1:1 and 1:50, between 1:1 and 1:250, including any range in between. As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

In some embodiments, partial degradation refers to a degradation of at least 10%, and between 10 and 90%, between, 10 and 20%, between 20 and 30%, between 15 and 25%, between 30 and 40%, between 40 and 50%, between 50 and 60%, between 60 and 70%, between 70 and 80%, between 80 and 90%, including any range in between. In some embodiments, degradation is assessed within a time period of up to 10 days, up to 5 days, up to 2 days, up to 1 day, including any range between.

In some embodiments, the location is selected from soil and a water source. Non-limiting examples of water sources are rivers, lakes, reservoirs, ponds, streams, ground water, spring water, sea, and ocean.

In another aspect, a method for degrading a crude oil or a crude oil fraction, comprising contacting the crude oil or the crude oil fraction with a composition comprising a carbon aerogel and a material bound to the carbon aerogel; wherein the material comprises a nitrogen-containing material and a phosphorus-containing material; wherein a combined content of nitrogen and phosphorus within the carbon aerogel is between 1 and 20 at %; and wherein the composition comprises oil degrading bacteria attached to the carbon aerogel.

In some embodiments, the combined content of nitrogen and phosphorus is sufficient for inducing increased degradation of the crude oil or of the crude oil fraction, as compared to a similar composition devoid of the material.

In another aspect a method for manufacturing the composition of the invention comprising (i) contacting cellulose-based material (CSB) with urea under condition sufficient for absorbing urea to the CSB, thereby obtaining CSB absorbed with urea (CSBU) (ii) contacting the CSB-N with phosphoric acid under appropriate conditions, thereby obtaining a CSB-N absorbed with phosphoric acid (CSB-NP) (iii) pyrolysis of the CSB-NP, thereby obtaining the aerogel of the invention.

In some embodiments, the condition sufficient for absorbing urea on the CSB (step i) are contacting at a temperature ranging between 15 and 60° C., between 15 and 25° C., between 2° and 30° C., between 30 and 40° C., between 4° and 50° C., between 5° and 60° C., including any range in between.

In some embodiments, the appropriate conditions in step (ii) are contacting the phosphoric acid with CSB-N at a temperature ranging from 20 and 150° C., 20 and 50° C., between 4° and 70° C., between 6° and 90° C., between 8° and 110° C., between 10° and 130° C., between 12° and 150° C., between 13° and 150° C., between 135 and 145° C., including any range in between.

In some embodiments, step (i) and step (ii) are sequential. In some embodiments step (ii) is prior to step (i). In some embodiments, step (i) and step (ii) cannot be performed simultaneously.

In some embodiments, the pyrolysis step (step iii), wherein the aerogel is heated at a temperature between 100 and 450° C., between 100 and 150° C., between 150 and 200° C., between 25° and 300° C., between 30° and 350° C., between 35° and 400° C., between 40° and 450° C., including any range in between. In some embodiments, the pyrolysis step (step iii), wherein pyrolysis is performed for a period of time of at most 6 hours, and between 30 min and 3 hours, 30 min and 1 h, between 1 and 2 h, between 2 and 3 h, between 3 and 4 h, between 4 and 5 h, between 5 and 6 h including any range in between.

In some embodiments, pyrolysis is performed at a temperature between about 400 and about 450° C. for a time period of at most 3 h, or between 0.1 and 3 h, between 0.5 and 3 h, between 1 and 3 h, including any range in between.

In some embodiments, prior to the pyrolysis step the method further comprises a preliminary degassing step, wherein degassing comprises subsequently subjecting the aerogel to vacuum and inert gas (e.g., a gas which is not oxygen, such as nitrogen, argon, etc.).

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Preparation of Carbon Aerogels

Filter paper scraps (500 mg) were put with 50 mL of double distilled water (DDW) and left still for 24 h. Then the scraps were washed several times with DDW to remove unwanted debris. The scraps were placed in a beaker with 30 mL of DDW, followed by strong agitation under vigorous magnetic stirring to form a uniform mixture which is called pulp. The pulp mixture was aliquoted to test tubes and the solids were concentrated by centrifugation. The supernatant was carefully removed. The pulp was then put into Eppendorf tubes and subjected to freeze-drying using Heto PowerDry PL 6000 Freeze Dryer (Thermo Fisher Scientific, USA) at −55° C. for 24 hours to form the desirable shaped pulp fiber aero-gel. After that, the pulp fiber aerogel was transferred into a Saftherm STG60-12 tubular furnace (Henan Sante Furnace Technology co., LTD, China) for pyrolysis. In order to completely remove the air trapped in the pulp fiber aerogel, the furnace was evacuated prior to the introduction of argon gas. This was followed by evacuation of the furnace again, repeated 3 times. After that, the furnace was heated up to 450° C. at a heating rate of 2.5° C. min-1 and kept at 450° C. for 3 h in vacuumed atmosphere (Pressure of ˜10-1 torr).

### Preparation of Nutrient Supplemented Carbonized Aerogels

Similar methodology was used to prepare the nutrient supplemented carbonized aerogel (NSCA). After the wet pulp was concentrated the nutrient incorporation process began. In a chemical hood, 50 mg of urea was put in a beaker and heated until molten. Then, the concentrated wet pulp (originally 500 mg as dry paper) was added to the beaker together with 5 μl phosphoric acid 85.3%. The mixture was kept at 140° C. and was constantly stirred for 30 minutes, to reach homogeneity. The heat was then lowered to 80° C. for another 60 minutes to ensure the evaporation of excess water. Then, the nutrient carrying pulp was divided into Eppendorf tubes and was freeze dried and proceeded on to the pyrolysis stage under the same conditions as described for the carbonized aerogel (CA).

### Aerogel Product Analysis

Temperature programmed desorption (TPD) analysis was carried out to test the thermal desorption process that occurs within each pulp material as it is slowly heated (4° C./min) from room temperature to 750° C. in an inert atmosphere (Ar). TPD analysis allows the determination of the suitable pyrolysis temperature based on the output spectra, using MAX300-EGA (Extrel CMS, LLC, USA), that indicates the temperatures at which sufficient oxygen ablation is achieved while nitrogen and phosphorous components are not entirely exhausted.

Products Elemental composition was examined via energy dispersive spectroscopy (EDS) analysis using FEI ESEM Quanta 200 scanning system.

This allowed the determination of the extent of carbonization as well as confirmation of nutrients' successful incorporation. This data was used for calculation of the yield on nutrient supplementation.

Aerogel sorbent properties were characterized in terms of hydrophobicity and adsorption capacities. Hydrophobicity is evaluated based on water contact angle using dataphysics OCA 20 system coupled with dataphysics SCA 20 software (DataPhysics Inst. GmbH, Germany) that calculates the angle that is formed between a single 5 μl water droplet and the aerogels surface.

Products surface area was measured according to the world-renowned Brunauer-Emmett-Teller (BET) theory using ASAP 2020 (Micromeritics Inst. Co., USA). BET theory suggests that surface area is best described as the external surface area of a solid object including surface attributable to pores based on gas adsorption. The BET analyzer calculates surface area from the estimated monolayer coverage of adsorbed gas.

### Nutrient Availability Test

To demonstrate the enhanced biodegradation induced by the nutrient carrying aerogel a bacteria model was put together. 10 ml of sterilized tape water was put into a test tube with 100 μl of light crude oil and 100 μl of bacteria starter in concentration of approx. 108 bacteria/ml. The bacteria used in these experiments were extracted from an environmental sample taken from oil contaminated soil and were of Pseudomonas Genus and Brucella Genus. 50 mg of NSCA was put on top the floating oil. There were 2 referential groups, both contained the same volume of water and oil. Control 1. (C1.) CA with bacteria. (C2.) No aerogel with bacteria. All of the experimental groups were incubated for 12 days on a rotating chamber in 30° C. The enhanced biodegradation model was evaluated in terms of crude oil residuals by Gas Chromatography-Mass Spectrometry (GC-MS) analysis using a 7890N GC connected to 5977A single-quadrupole mass-selective detector.

### Bacterial Adhesion Observations

Biofilm formation was observed using FEI ESEM Quanta 200 scanning electron microscopy (SEM). Aerogels were collected after 12 days of incubation and the samples were prepared for SEM studies as follows. Initially, the samples were carefully washed with hexane to extract crude oil. After fixation in 2.5% buffered glutaraldehyde, the samples were subsequently dehydrated via an ascending, serial ethanol gradient and immersed in a hexamethyldisilazane (HMDS)/ethanol gradient solution (25%, 50%, 75%, 90%, 95% and 100%). The treated specimens were air dried for 4 hours, and in preparation for SEM scanning, they were sputter coated with a 20 nm layer of gold using the EMITECH K575x sputtering device (Emitech Ltd, UK).

### Example 2

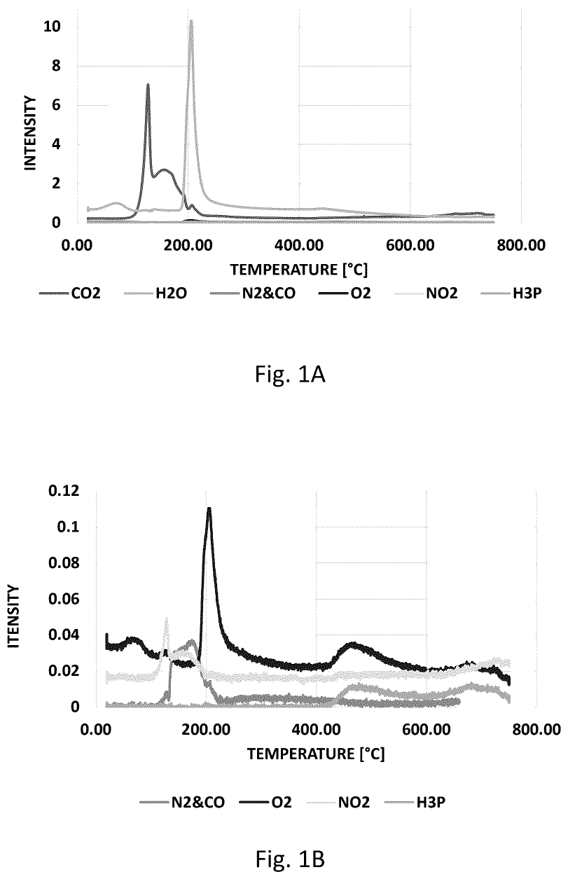

Aerogel products post pyrolysis significantly vary with respect to pyrolysis conditions such as pyrolysis temperature, atmosphere and duration. Carbonized aerogels are commonly produced under vacuum to avoid pressure induced stress and to allow the formation of the ultralight and porous 3D network. In this research pyrolysis temperature was selected to suit the goal of carbonization combined with nutrient incorporation. FIG. 1 presents the TPD spectra of the exhaust stream from which thermal desorption of oxygen containing gases can be observed in the intention to determine the optimal temperature for desired anaerobic combustion. As mentioned previously, the target pyrolysis temperature allows the balance between extensive oxygen release and sufficient nutrients yield.

Typically, combustion of organic materials results mainly in the release of CO2 and H2O. TPD spectra of the exhaust stream indicated that these gases peak at 125° C. and 205° C. respectively. A deeper look on the less abundant gases in the exhaust stream shows a small peak at approximately 450° C. after which oxygen release is decreased. Further release of NO2 and H3P at temperatures higher than 450° C. indicates that indeed anaerobic combustion at this target temperature may result in successful incorporation of nitrogen and phosphorus moieties. Thus, the pyrolysis process was set to start with slow heating to 450° C. at a rate of 2.5° C. per minute to ensure evaporation of residue moisture as well as to prevent extreme chemical reactions within the pulp fibers. Suitable time at set temperature was found to be 3 h as a function of carbonization and mass loss. Shorter periods at set point yielded products that were relatively high in oxygen and thus exhibited low to zero hydrophobicity. On the other hand, the inventors found that longer periods at 450° C. yielded in extreme mass loss (˜98%) as the pulp gradually became perished.

Chemical composition of the aerogel products is examined in terms of total elemental relative abundance. EDS analysis does not allow for the detection of Hydrogen, and focuses on the most abundant elements in the aerogel's structure. The carbonized aerogel precursor is pure cellulose paper, containing Hydrogen Carbon and Oxygen, and the introduction of Nitrogen and Phosphorus elements occurs via the supplementation of nutrients. Relevant elemental compositions are described in table 1.

TABLE 1
EDS analysis - product element composition

| | % C | % O | % N | % P |
|---|---|---|---|---|
| Cellulose Paper | 55 ± 0.4 | 45 ± 0.5 | — | — |
| Carbonized Aerogel | 94 ± 0.2 | 6 ± 0.8 | — | — |
| Nutrient Supplemented | 85 ± 2.9 | 10 ± 2.3 | 3 ± 1.0 | 2 ± 0.8 |
| Carbonized Aerogel | | | | |

Compared to natural cellulose, CA is comprised mainly of Carbon, indicating the extensive decomposition of oxygen from within the cellulose fibers during combustion. Oxygen levels are slightly elevated by the supplementation of nutrients as oxygen mediates the intermolecular bonds between the three reagents. For both aerogel types, under the specified pyrolysis conditions combustion consumes 91%±2% of the original cellulose pulps' weight. It was found that for NSCA the yield on Phosphorous (˜73%) was significantly higher than that of Nitrogen (˜20%). This result is correlated with TPD chart (FIG. 1) that shows peaks of NO2 at lower combustion temperatures relative to those observed for H3P gas which occur at higher temperatures. This further strengthens the concept that during the phosphorylation of cellulose in the presence of urea, phosphoric acid directly binds to cellulose fibers while urea is bonded to the complex via phosphoric acid.

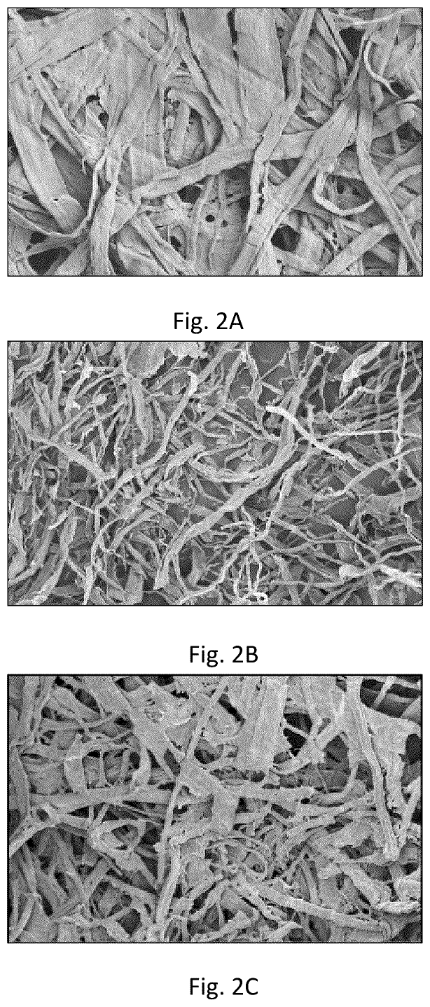

Further effects of supplemented nutrients on post pyrolysis products were evaluated through SEM imaging. Herein (FIG. 2) it is shown that NSCA fibers are found to be denser compared to CA. BET analysis supports these findings as it indicated that NSCA aerogels has surface area of 217.9±5.3 m2/g, lower by twofold compared to CA's surface area of 438.3±8.9 m2/g. This difference in the products' porosities and light structure may result from heat retardance effect caused by the energetic uptake of the added nutrients. The supplemented nutrients are clearly seen upon the cellulose fibers as thin interconnected inhomogeneous web (FIG. 2).

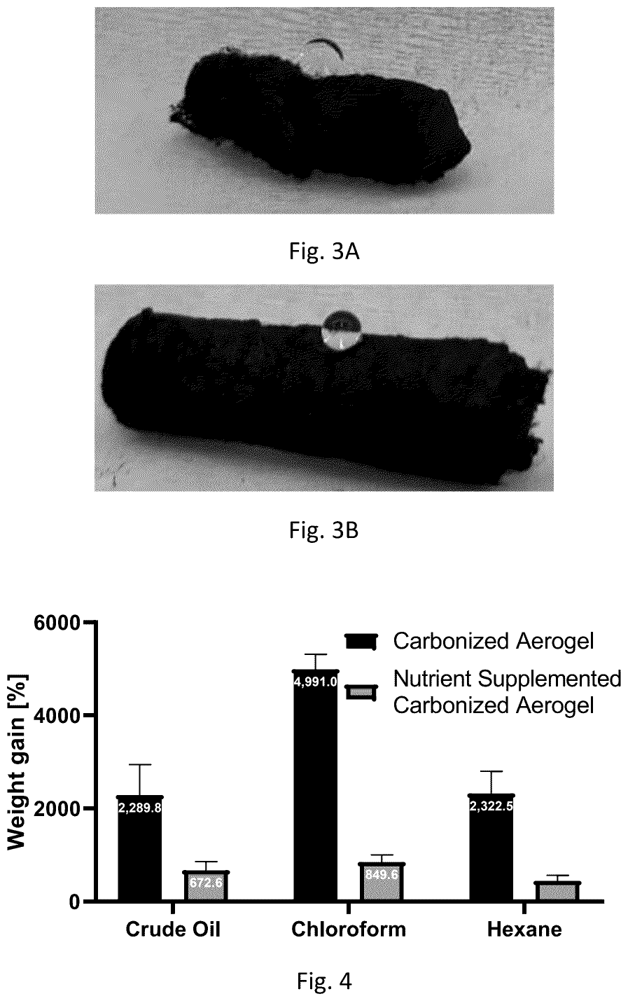

The aerogel is evaluated as a hydrophobic sorbent, particularly due to its hydrophobic nature, expressed by its ability to repel water. FIG. 3 exhibits the high hydrophobicity of both of the examined aerogel products.

Hydrophobicity can be described as a quantitative parameter, characterized by the contact angle observed between the circular water droplet and the aerogel's flattened surface. Contact angle measurements indicated similar hydrophobicity levels of ˜131±3° which are considered as highly hydrophobic. Therefore, there was no influence of nutrients supplementation on the aerogel's hydrophobicity.

Adsorption capacities of organic solvents from the aqueous environments are highly important in the examination of the applicability of a sorbent. Given the aforementioned high hydrophobicity of the aerogel, adsorption capacities of organic solvents were calculated with negligible amount of adsorbed water (˜1 mg water/mg aerogel). FIG. 4 summarizes weight gain as it represents the amounts of organic substances adsorbed onto acrogels.

Three substances were chosen to test adsorption capacity, as each represents a class of common pollutants. Crude oil, Chloroform (CHCl3) and Hexane (C6H14). Adsorption of hexane indicates the potential of the sorbent to adsorb a large range of linear alkanes. These substances are all of hydrophobic nature and prone to interact with non-polar molecules.

It is shown in FIG. 4 that adsorption of all three substances was dramatically reduced (by 3-6 folds) by the supplementation of nitrogen and phosphorous. Significant adsorption capacities are dependent on the porosity of the sorbent. Thus, as the denser NSCA was found to be less porous, its adsorption potential becomes lower compared to the more porous CA. Lower porosity and the abundance of polar nitrogen and phosphorous moieties have a synergistic effect on the NSCA adsorption properties.

The influence of the utilization of NSCA in an oil biodegradation model is examined with reference to natural medium (sterile tap water) that does not involve the introduction of aerogel. Further comparisons were made with samples treated with CA as it was also found to promote oil biodegradation.

Pseudomonas and Brucella genus are frequently found in oil contaminated areas, as both exhibit oil degradation. The first observations of the enhanced degradation model indicated that the inoculation of bacteria (Pseudomonas and Brucella) to the aerogel containing media was coupled with aerogels apparent descent after several days. Thus, it was presumed that aerogel allows for bacterial adhesion upon its fibers and allows for the development of a biofilm.

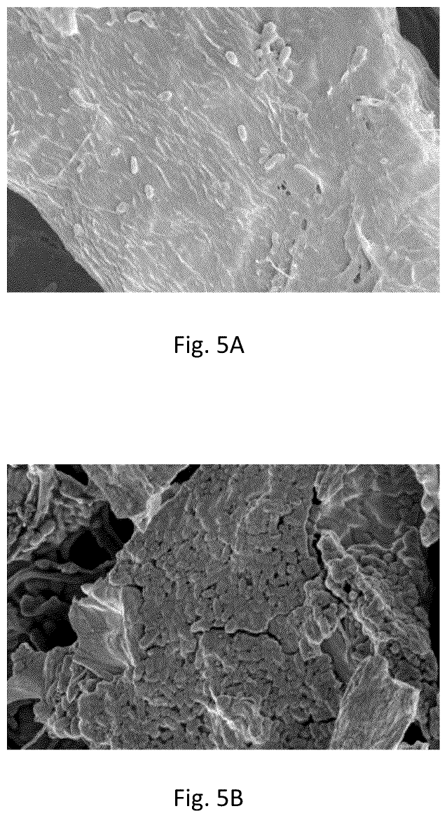

In all cases of aerogel treatments, oil was adsorbed immediately. No difference in adsorption was observed between samples containing CA or NSCA. Moreover, in cases where aerogels were combined with oil degrading bacteria, three days into the experiment, the aerogels sank to the bottom of the tubes. This was in contrast to the cases where bacteria were not introduced into the medium. FIG. 5 shows the development of biofilms around and within the aerogel fibers. Naturally, biofilms are comprised of adherent cells that become embedded in the fibers through extracellular polymeric substances that are largely composed of highly hydrated biopolymers. Thus, the aerogel-oil-biofilm combination which is heavier than the aerogel alone becomes surrounded by biopolymers that lower the water repulsion causing the aerogel to gradually sink. In future applications of NSCA for oil biodegradation in-situ, this matter should be addressed. It is evident that the use of NSCA allows for greater proliferation of bacterial culture upon the aerogel's fibers compared to the use of CA (FIG. 5). Bacterial adhesion at maximal surface density is presented in FIG. 5.b. Without being bound to any theory, the inventors propose that as oil becomes adsorbed into pores of the sorbent material, direct contact between oil and supplemented nutrients is carried out by bacterial cells that adhere to the aerogel and so may promote enhanced biodegradation. This indicates that due to the bacterial nutrients present in the aerogel a highly efficient bacterial adhesion occurs, the strategy presented in the present work results in a more efficient aerogel when compared to bacterial immobilization alone. This shows the potential promotion of the proliferation of microorganisms that are endogenous in a polluted environment rather than introduction of foreign bacteria.

The biodegradation model was further examined as oil residual post-treatment analysis using Gas Chromatography-Mass Spectrometry (GC-MS). In order to simulate the natural occurrence of oil biodegradation in aqueous environments with diverse microbial populations, a preliminary experiment was carried out comparing oil degradation treatment using bacteria from two different genera: Pseudomonas and Brucella and their combination.

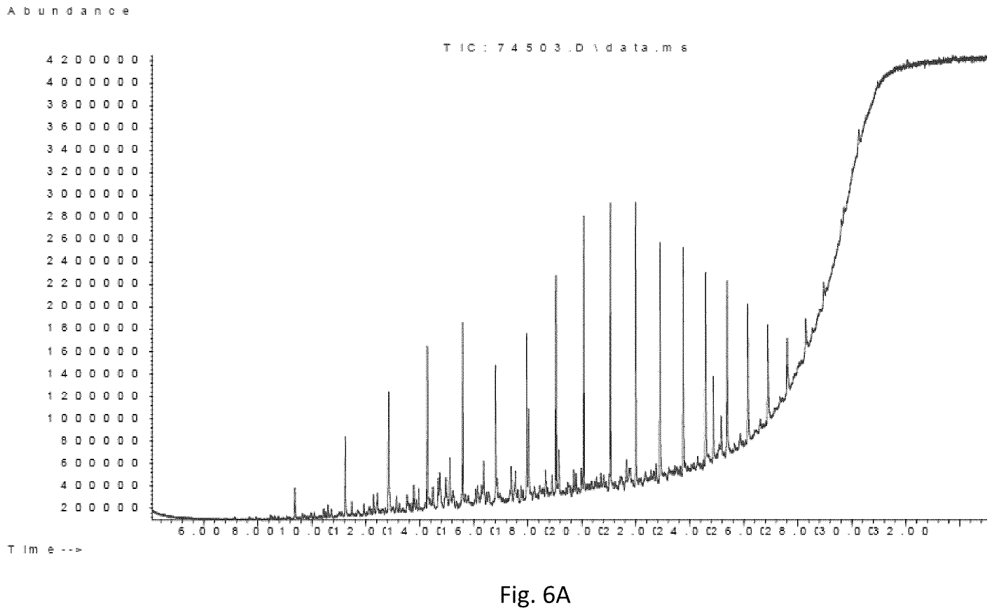

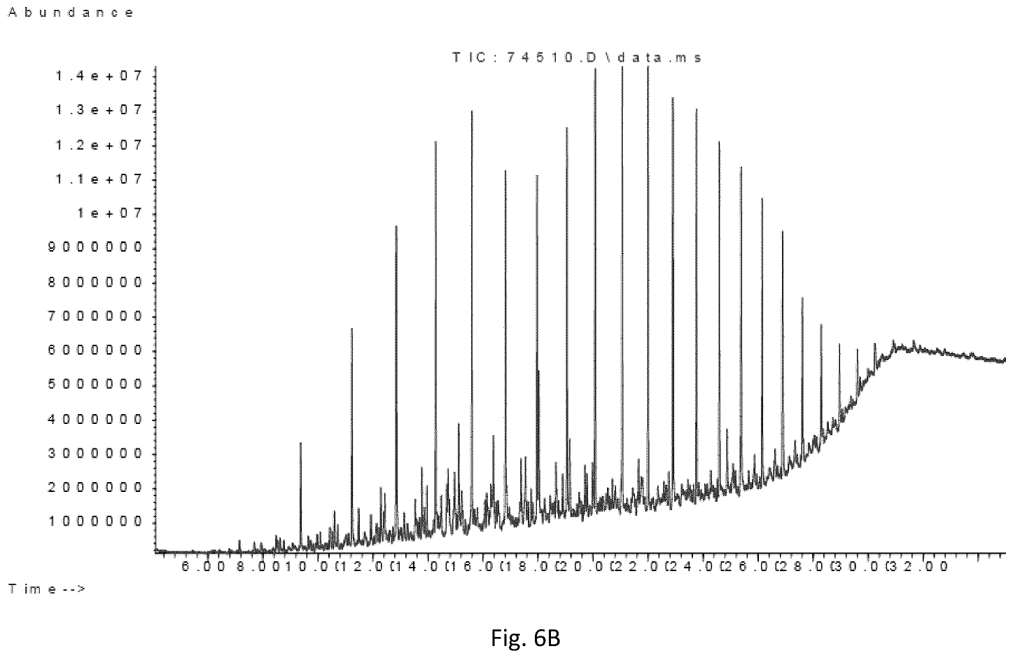

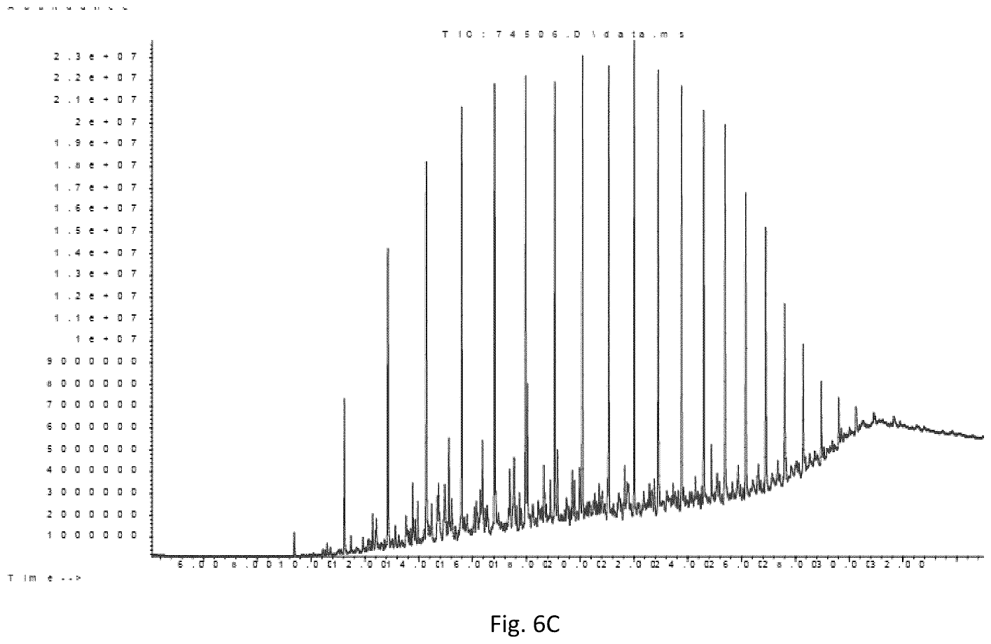

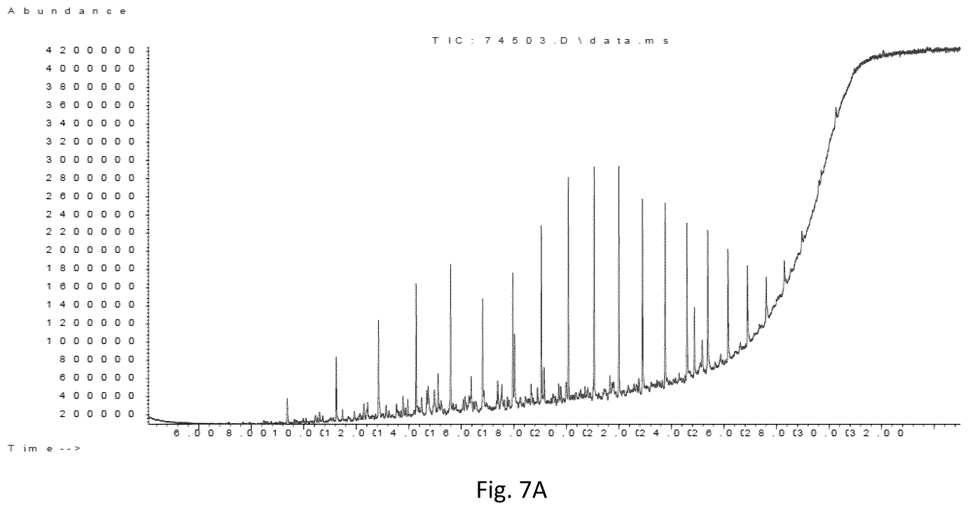

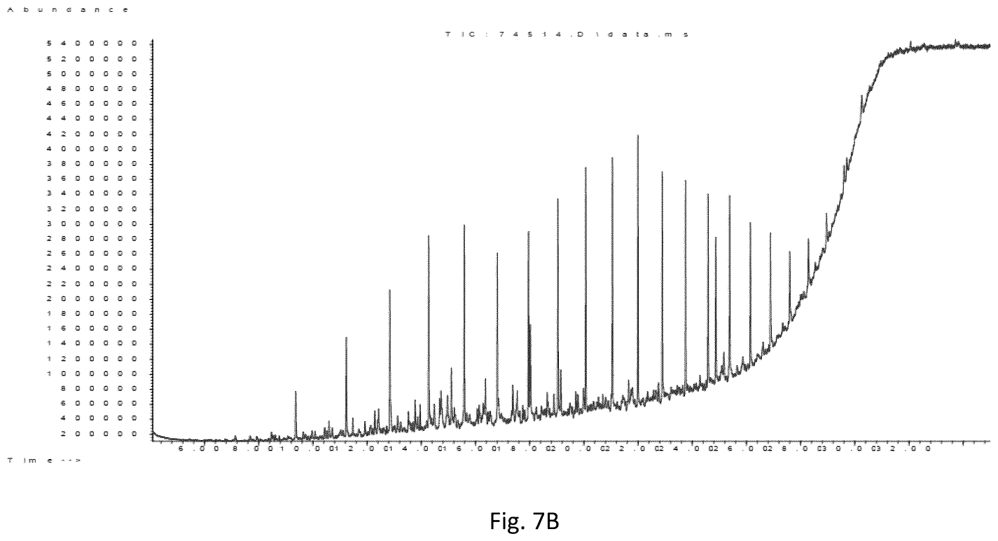

GC-MS chromatograms (FIG. 6) show the difference in oil residual post treatment using NSCA. Maximum abundance levels were found to be ˜3.0*106 for oil treated with mixed culture of Pseudomonas & Brucella; ˜1.4*107 for oil treated with Pseudomonas; and ˜2.3*107 for oil treated with Brucella. Data from GC-MS reports was processed to allow comparative examination of different oil component concentrations. The relative change in each identified oil component post treatment was calculated. These calculations indicated that oil degradation of samples treated with the mixed culture was more extensive after 12 days (using 0.5 g aerogel per g oil, in 10 ml medium, 10\hat{ }7 cfu/ml) by 86% and 90% on average, compared to oil degraded by isolated Pseudomonas and isolated Brucella respectively. Biodegradation using a consortium of microorganisms can result in minimal oil residue compared to individual strains. This is compatible with natural bacterial diversity found in contaminated water or soil. After the superior efficiency exhibited by the use of mixed culture over bacteria isolates was established, further experimentation evaluated the influence of aerogel utilization on the extent of biodegradation.

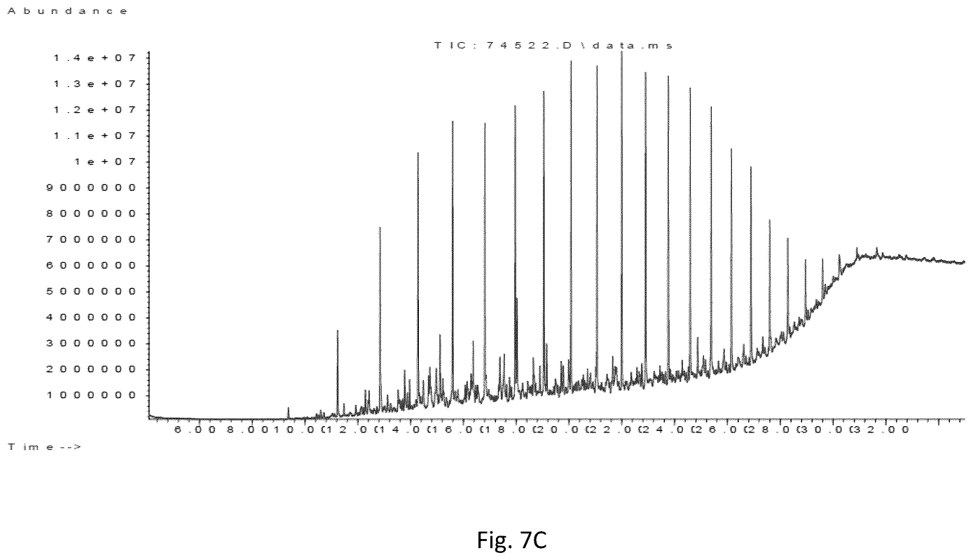

GC-MS analysis examined the difference in oil residuals post biodegradation treatment using mixed bacterial culture containing equal amounts of bacteria of Pseudomonas and Brucella genera (FIG. 7). Maximum abundance levels were recorded as ˜1.4*10 s⁷ for samples treated with no aerogel; ˜4.2*106 for samples treated with CA; and 3.0*106 for samples treated with NSCA. This confirmed that oil biodegradation was most efficient when NSCA was introduced into the media. Enhanced oil degradation was achieved when NSCA was utilized as the average decrease in oil's components was higher by 52% and by 85% compared to samples contained CA and with no aerogel, respectively. It is presumed C:(N+P) higher biodegradation efficiencies can be met with supplementation of nutrient at stoichiometric ratio of between 100:5 and 100:20.

The higher degradation rate achieved with the utilization of NSCA is a proof of concept that nutrients can be bioavailable when supplemented via incorporation into the cellulose-based aerogel. These results are correlated with the visually more massive bacterial adhesion on the aerogel fibers.

The inventors had shown that NSCA can enhance biodegradation by increasing breakdown of crude oil components as compared to CA alone or to no aerogel at all. This indicates that the process of nutrient supplementation yielded a bioavailable source of nitrogen and phosphorous for bacteria to utilize in order to biodegrade the hydrocarbons from the water column. It is also suggested that the aerogels may serve as an adhesion surface that promotes biofilm formation. Furthermore, the aerogel act by adsorbing and therefore concentrating oil within its fibers. This allows for contact between the adsorbed hydrocarbons, the supplemented nutrients and the oil degrading bacteria.

The observed that upon absorbance of crude oil within the aerogel the density of the aerogel increases, thus inducing sinking.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising a carbon aerogel and a material bound to said carbon aerogel; wherein:
said material comprises a nitrogen-containing material and a phosphorus-containing material; wherein the combined content of nitrogen and phosphorus within said composition is at least 1 atomic percentage (at %); wherein said material further comprises an additional element selected from Fe, Zn, S, Na, Ca, Ti, and Mg, including any salt, any oxide, any complex, or any combination thereof.

2. The composition of claim 1, wherein said aerogel is characterized by carbon content between 70 and 95 at %.

3. (canceled)

4. The composition of claim 1, wherein said additional element within said composition is present at a concentration of 0.1 to 10 at %.

5. The composition of claim 1, wherein said carbon aerogel is in a form of a matrix comprising a plurality of carbon fibers.

6. (canceled)

7. The composition of claim 1, wherein said carbon aerogel is characterized by: (i) a nitrogen content of between 1 and 10 at %; (ii) a phosphorus content of between 1 and 10 at %, or both (i) and (ii).

8. The composition of claim 1, wherein said carbon aerogel is further characterized by an oxygen content of between 5 and 20 at %.

9. The composition of claim 1, wherein said carbon aerogel is characterized by a water contact angle of between 12° and 140°.

10. The composition of claim 1, wherein said carbon aerogel is characterized by porosity of between 95 and 99%.

11. The composition of claim 1, further comprising bacterial cells attached to said carbon aerogel.

12. The composition of claim 11, wherein said bacterial cells are oil degrading bacteria.

13. The composition of claim 12, wherein said oil degrading bacteria comprise one or more bacterial species selected from genera Pseudomonas and Brucella.

14. (canceled)

15. The composition of claim 1, wherein said carbon aerogel is capable of absorbing at least 100% of a water immiscible organic compound by weight of said carbon aerogel.

16. The composition of claim 15, wherein said water immiscible organic compound is selected from crude oil, a crude oil fraction and a water immiscible organic solvent.

17. The composition of claim 1, wherein said carbon aerogel is characterized by BET surface area of between 150 and 300 m²/g.

18. A method for removing a crude oil or a crude oil fraction, comprising applying the composition of claim 1 to said crude oil or said crude oil fraction, thereby removing said crude oil or said crude oil fraction.

19. The method of claim 18, wherein said composition comprises oil degrading bacteria attached to the carbon aerogel.

20. The method of claim 18, wherein said applying is at an amount sufficient for substantially absorbing said crude oil or said crude oil fraction.

21. The method of claim 18, wherein said crude oil or said crude oil fraction is present in soil or water.

22. A method for degrading a crude oil or a crude oil fraction, comprising contacting the crude oil or the crude oil fraction with a composition comprising a carbon aerogel and a material bound to said carbon aerogel; wherein said material comprises a nitrogen-containing material and a phosphorus-containing material; wherein a combined content of nitrogen and phosphorus within said carbon aerogel is at least 1 at %; and wherein said composition comprises oil degrading bacteria attached to said carbon aerogel.

23. The method of claim 22, wherein said combined content of nitrogen and phosphorus is sufficient for inducing increased degradation of said crude oil or of said crude oil fraction, as compared to a similar composition devoid of said material.
